(19) United States
(12) Patent Application Publication
(10) Pub. No.: US 2021/0322207 A1
(43) Pub. Date: Oct. 21, 2021

(54) POSTOPERATIVE FACIAL COOLING PACK

(71) Applicant: Annesleigh Hope Bengtson, Greensboro, NC (US)

(72) Inventor: Annesleigh Hope Bengtson, Greensboro, NC (US)

(21) Appl. No.: 17/221,980

(22) Filed: Apr. 5, 2021

Related U.S. Application Data
(60) Provisional application No. 63/010,914, filed on Apr. 16, 2020.

(51) Int. Cl. A61F 7/02; A61F 7/10

(52) U.S. Cl. CPC A61F 7/02; A61F 7/10; A61F 2007/0003; A61F 2007/023; A61F 2007/0285

(57) ABSTRACT

A modular cooling pack and method of cooling a head of a patient includes a first pocket, a first cooling capsule, a second pocket, a second cooling capsule, a third pocket, a third cooling capsule, and a securement strap. Each of the first, second, and third cooling capsules are respectively configured to be received within the first, second, and third pockets, cooled, and applied to the head of the patient. The securement strap is configured to receive the first, second, and third pockets thereagainst and at least partially define a closed loop configured to be received about the head of the patient to thereby removably secure the first, second, and third pockets against the head of the patient in at least one of a plurality of assembled configurations.

### CROSS-REFERENCE TO RELATED APPLICATION

This application claims priority to U.S. Pat. App. No. 63/010,914, filed on Apr. 16, 2020, entitled “Postoperative Facial Cooling Pack,” which is hereby incorporated by reference herein in its entirety.

### TECHNICAL FIELD

The present invention relates generally to devices and methods for facilitating facial healing, and more particularly, to a modular cooling pack or cooling pack for postoperative facial healing and related methods.

### BACKGROUND

Cryotherapy or cold therapy is widely used for promoting physical healing by applying local cold temperatures to a particular area of a patient's body. Cold therapy is commonly used to promote facial healing, such as during facial surgery recovery or dermatological treatment, by applying one or more cooling packs to the patient's face. In this regard, millions of people undergo various types of facial surgery each year, including dental/oral surgical procedures such as wisdom tooth removal or upper jaw surgery and cosmetic/plastic surgical procedures such as rhinoplasty (i.e., nose reshaping) or eyelid surgery. Cooling packs are routinely applied to the faces of patients recovering from such procedures, undergoing various types of facial dermatological treatment, or undergoing any other type of facial healing regimen.

Current cooling packs suffer from numerous disadvantages that result in undesirable patient discomfort. For example, when applied to a patient's face, cooling packs frequently extend beyond the targeted treatment site (e.g., the patient's upper jaw) and contact other portions of the patient's face (e.g., the patient's nose) thereby causing unnecessary cooling of such other portions in a manner that may chill the patient. Cooling packs also frequently extend beyond the targeted treatment site and undesirably obstruct the patient's mouth and/or nostrils in a manner that may interfere with the patient's ability to breathe. In some cases, the contents within a cooling pack may gradually melt and leak out of the cooling pack onto the patient's face, resulting in further patient discomfort and requiring a separate cloth to absorb the melted material from the patient's face. Moreover, traditional cooling packs simply rest on the patient's face in an unsecured manner such that the patient is required to lay on the patient's back thereby restricting the patient's mobility while wearing the cooling pack.

Medical practitioners such as oral surgeons, plastic surgeons, dermatologists, and other facial healing practitioners, patients of such practitioners, and medical supplies manufacturers and distributors continue to seek improved cooling packs. Accordingly, there is a need for a modular cooling pack for facial healing and related methods that address the present challenges such as those discussed above.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a front elevational view of an example of a modular cooling pack in an exemplary disassembled configuration;

FIG. 2 depicts a perspective view of the modular cooling pack of FIG. 1 in a first exemplary assembled configuration;

FIG. 3 depicts a perspective view of a round pocket and a round cooling capsule of the modular cooling pack of FIG. 1;

FIG. 4 depicts a perspective view of an elongate pocket and an elongate cooling capsule of the module cooling pack of FIG. 1;

FIG. 5A depicts a side perspective view of the modular cooling pack of FIG. 1 in the first exemplary assembled configuration positioned on a head of a patient for promoting facial healing during upper jaw surgery recovery;

FIG. 5B depicts a side perspective view of the modular cooling pack of FIG. 1 in a second exemplary assembled configuration positioned on a head of a patient for promoting facial healing during rhinoplasty recovery;

FIG. 5C depicts a side perspective view of the modular cooling pack of FIG. 1 in a third exemplary assembled configuration positioned on a head of a patient for promoting facial healing during wisdom tooth removal recovery; and

FIG. 5D depicts a front perspective view of the modular cooling pack of FIG. 1 in a fourth exemplary assembled configuration positioned on a head of a patient for promoting pain relief from a headache.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Modular Cooling Pack for Facial Healing

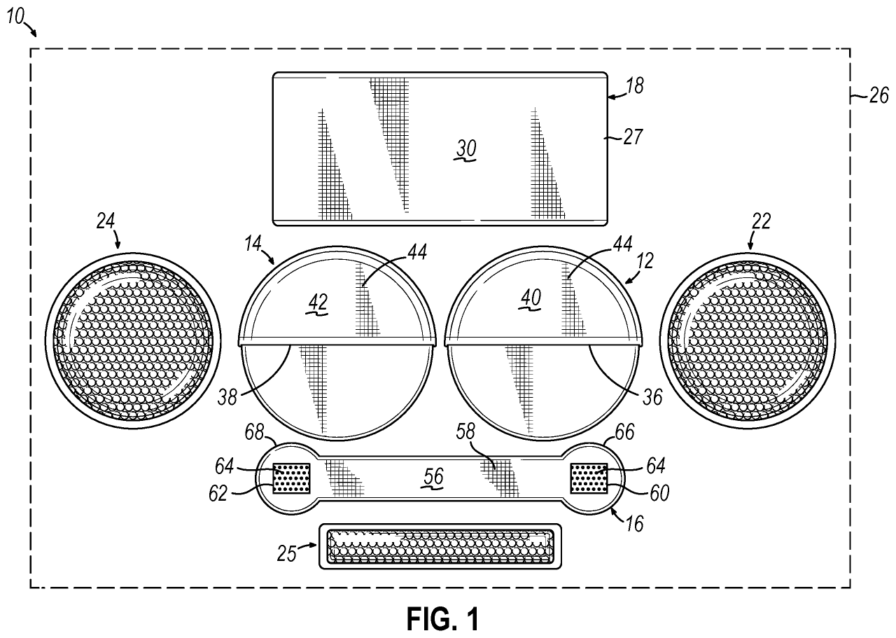

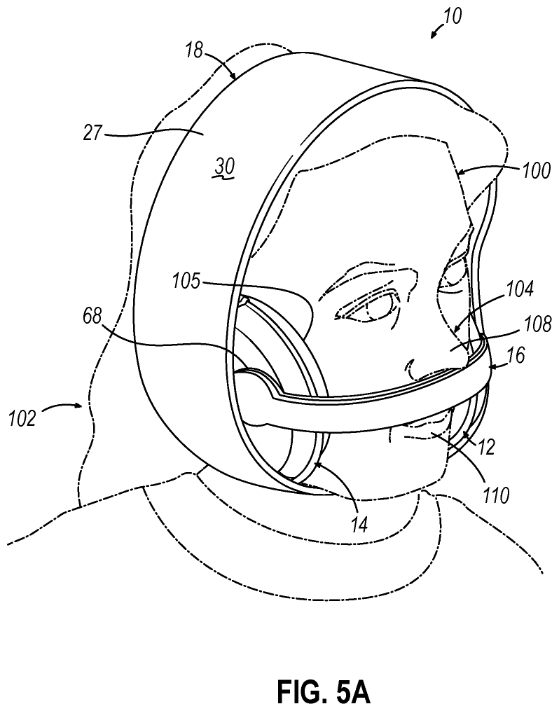

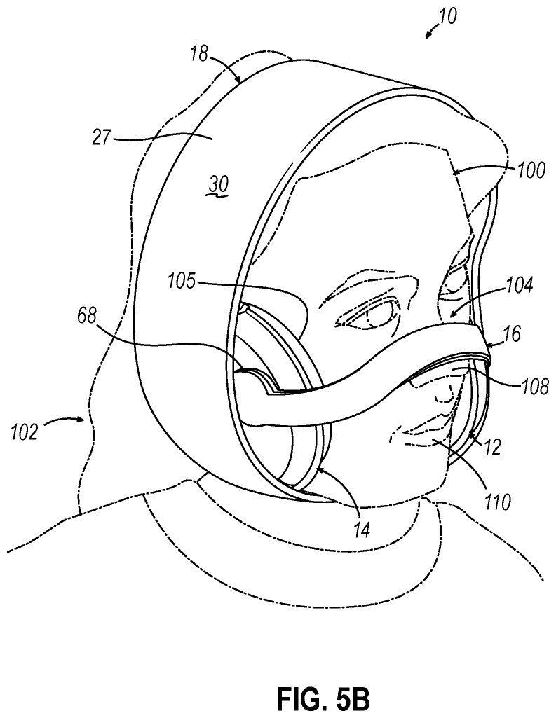

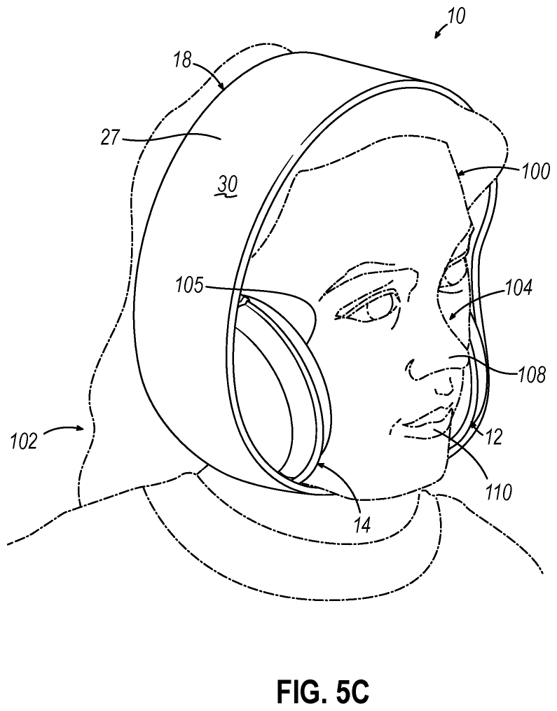

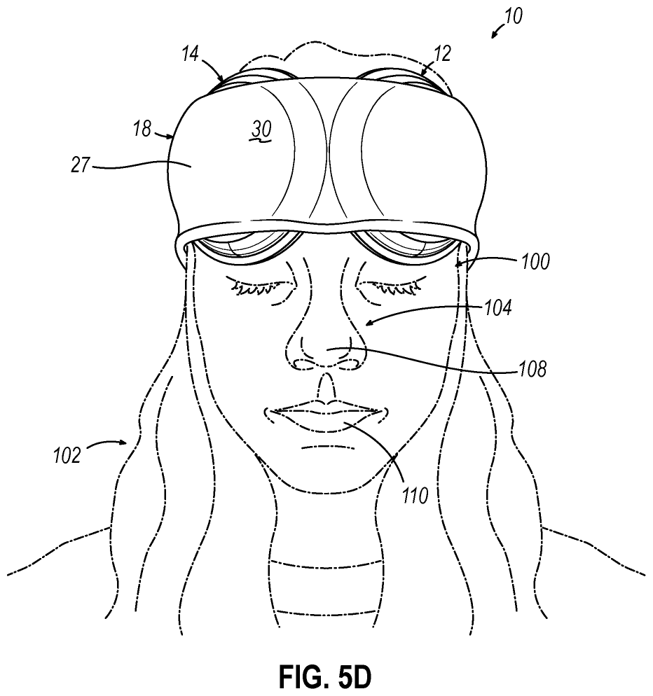

FIG. 1 shows an example of a modular cooling pack (10) for head pain relief, including facial healing. The present example of modular cooling pack (10) includes a plurality of selectively attachable components, including left and right generally round pockets (12, 14), a generally elongate pocket (16), a securement strap (18), left and right generally round cooling capsules (22, 24), and a generally elongate cooling capsule (25). As described in greater detail below, modular cooling pack (10) is configurable in a plurality of different configurations (see FIGS. 5A-5D) to provide increased comfort, compliance, and convenience for relieving head pain, including promoting facial healing. As used herein, the terms “left” and “right” are merely relative terms described with respect to the patient when positioned on the patient in use and are not intended to unnecessarily limit the invention.

FIG. 1 more particularly shows modular cooling pack (10) in an exemplary disassembled configuration such that each of pockets (12, 14, 16), securement strap (18), and cooling capsules (22, 24, 25) are spaced apart and disconnected from each other. Alternative disassembled configurations may have one or more of pockets (12, 14, 16), securement strap (18), and cooling capsules (22, 24, 25) at least partially, including fully, connected together such that the disassembled configuration is not intended to be unnecessarily limited to all of pockets (12, 14, 16), securement strap (18), and cooling capsules (22, 24, 25) being spaced apart and disconnected from each other in the arrangement shown in FIG. 1. Pockets (12, 14, 16), securement strap (18), and cooling capsules (22, 24, 25) may collectively be referred to herein as a “kit cooling pack” collected and combined into a packaging (26) for one or more arrangements of use for a patient. In addition, while pockets (12, 14) with cooling capsules (22, 24) are shown as round and pocket (16) with cooling capsule (15) is shown as elongate and generally formable to a linear shape, the invention is not intended to be unnecessarily limited to the round and elongate shapes shown in the present example and may vary so as to assemble for cooling as desired.

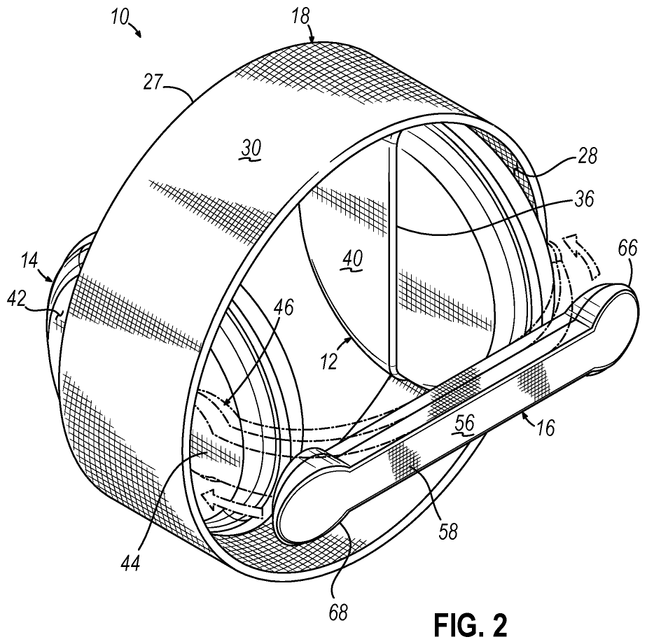

Securement strap (18) more particularly includes a band body (27) in the shape of a closed loop as shown in the present example in FIG. 2. Band body (27) has inner and outer surfaces (28, 30) that define a length, which may also be referred to herein as a circumference of the closed loop, and a width. Generally, the length is greater than the width such that the band body (27) wraps around, such as surrounding, a head of the patient to apply compression about, which may also be referred to as around, an entire circumference of the head. In turn, width of band body (27) is sized to capture one or more of pockets (12, 14, 16) with cooling capsules (22, 24, 25) therein between the head of the patient and inner surface (28) of band body (27). Band body (27) as shown is generally elastic and flexible in order to accommodate various head sizes, head shapes, and configurations of pockets (12, 14, 16) with cooling capsules (22, 24, 25) about the head. In one example, band body (27) is formed from a cotton material and has an unstretched length of approximately 8 inches and an unstretched width of approximately 3.5 inches. In any case, band body (27) in use elastically enlarges so as to be resiliently biased and in tension thereby applying compression about the head for releasable securement of one or more of pockets (12, 14, 16) with cooling capsules (22, 24, 25) against the head.

Alternatively, another example of a securement strap (not shown) may include an elongate fastening strap (not shown) with an outer surface having selective coupling features positioned thereon for selectively attaching elongate pocket (16) to various other components of modular cooling pack (10). In this regard, the fastening strap (not shown) includes a pair of patches substantially covered by hooks of a hook and loop fastener positioned at or near respective terminal ends thereof to attach to loops on other pockets (12, 14). Elongate fastening strap (not shown) thus may be shaped as an open loop and attached to one or more pockets (12, 14, 16) to form a closed loop compressing one or more pockets (12, 14, 16) against the head. It will thus be appreciated that securement strap may be a closed or open loop in alternative examples such that the invention is not intended to be unnecessarily limited to securement strap (18) shown in the present example.

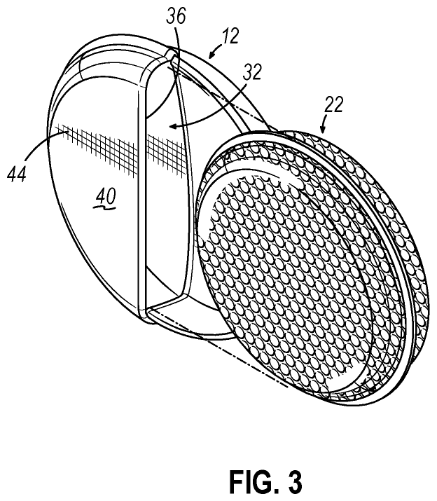

With respect to FIGS. 2-3, left and right round pockets (12, 14) include left and right interior cavities (34) sized and configured to selectively receive left and right round cooling capsules (22, 24), respectively. In one example, left and right round pockets (12, 14) further include respective closure flaps (36, 38) for inhibiting cooling capsules (22, 24) from becoming inadvertently dislodged from interior cavities (32). In any event, left and right round pockets (12, 14) also each include outer surfaces (40, 42) having selective coupling features positioned thereon for selectively attaching left and right round pockets (12, 14) to various other components of modular cooling pack (10). In this regard, outer surfaces (40, 42) of left and right round pockets (12, 14) shown in the present example are substantially covered by hook-catching loops (44), which may also be referred to as piles, of a hook and loop fastener (46) (e.g., VELCRO™). In one example, left and right round pockets (12, 14) are each constructed of a loosely-woven material, such as a polyester loop fleece fabric material, presenting loops (44) such that loops (44) are integral and unitarily formed with bodies of pockets (12, 14). In another example, such loops (44) may be discrete features attached to bodies of left and right round pockets (12, 14). While the present example shows left and right pockets (12, 14) as round with a diameter of approximately 4 inches, it will be appreciated that pockets in other examples may be alternatively shaped to be received against the head of the patient such that the invention is not intended to be unnecessarily limited to round pockets (12, 14) shown in the present example.

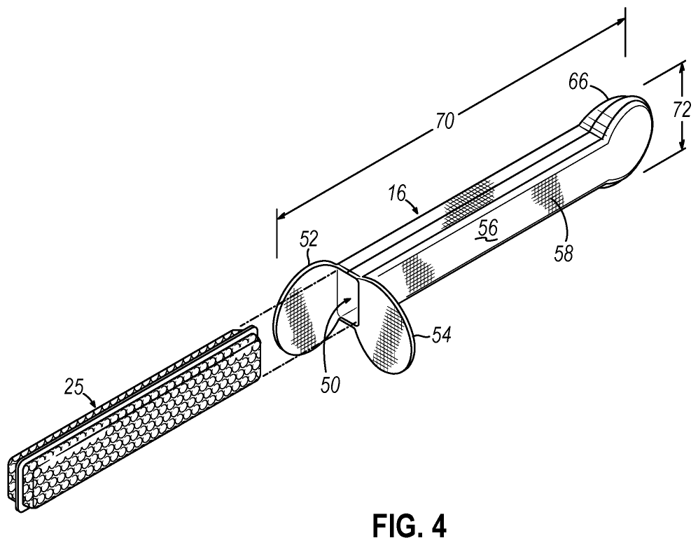

Similarly, FIGS. 2 and 4 show elongate pocket (16) including an elongate interior cavity (50) sized and configured to selectively receive elongate cooling capsule (25). In one example, elongate pocket (16) includes a closure flap (54) for inhibiting such a cooling capsule from becoming inadvertently dislodged from elongate interior cavity (50). Elongate pocket (16) is sized such that a width of elongate pocket (16) is substantially equal to or slightly less than a distance between a patient's nose and mouth such that elongate pocket (16) may be capable of extending across the patient's face between the patient's nose and mouth without interfering with the patient's ability to breathe through the patient's mouth or nostrils; and/or such that the width of elongate pocket (16) is substantially equal to or slightly less than a length of a patient's nose such that elongate pocket (16) may be capable of extending across the patient's face over the patient's nose without interfering with the patient's ability to breathe through the patient's nostrils.

In any event, elongate pocket (16) also includes an outer surface (56) having selective coupling features positioned thereon for selectively attaching elongate pocket (16) to various other components of modular cooling pack (10). In this regard, outer surface (56) of the present example of elongate pocket (16) is substantially covered by hook-catching loops (58). In one example, elongate pocket (16) may be constructed of a loosely-woven material presenting such loops (58). In addition, pocket (16) further includes left and right patches (60, 62) substantially covered by hooks (64) of hook and loop fasteners (46) positioned at or near respective left and right terminal end portions (66, 68) of elongate pocket (16). In one example, such as shown in FIG. 4, elongate pocket (16) is formed from a polyester loop fleece fabric material, has an elongate length (70) of approximately 6 inches, and a width (72), which is configured to extend transverse to elongate length (70) and between the nose and the mouth of the patient, of approximately 0.75 inches.

With respect to FIGS. 1-4, left and right round cooling capsules (22, 24) and elongate cooling capsule (25) of the present example contain refrigerant gel, such as gel beads, configured to be cooled to a desirably cold temperature, applied to the head for use, gradually warmed during use, and re-cooled for future reuse. However, it will be appreciated that capsules (22, 24, 25) may be filled with any material suitable for freezing or otherwise exhibiting relatively cold temperatures, such as water, clay, or other cooling packs, including packs with chemicals exhibiting endothermic chemical reactions for cooling. The invention is thus not intended to be unnecessarily limited to any particular contents in cooling capsules (22, 24, 25). In one example, left and right round cooling capsules (22, 24) and/or elongate cooling capsule (25) each include an outer surface having selective coupling features (not shown) positioned thereon for selectively attaching left and right cooling capsules (22, 24) and/or elongate cooling capsule to various other components of modular cooling pack (10). For example, left and right round cooling capsules (22, 24) may each include a respective patch (not shown) substantially covered by hook-catching loops.

Any of aforementioned loops (44, 58) may selectively catch or otherwise engage any of aforementioned hooks (64) to thereby selectively couple the respective components of modular cooling pack (10) to each other. More particularly, hooks (64) on left and right terminal end portions (66, 68) may releasably attach to left and right pockets (12, 14) in a variety of configurations. Furthermore, securement strap (18) may receive one or more of left and right pockets (12, 14) and left and right terminal end portions (66, 68) and compress one or more of left and right pockets (12, 14) and left and right terminal end portions (66, 68) against the head of the patient to further secure pockets (12, 14, 16) relative to each other about the head for use.

By way of further example, loops (44, 58) of left and right round pockets (12, 14) may selectively catch hooks (64) of elongate pocket (16) and/or hooks (not shown) on fastening strap (not shown) if applicable to thereby selectively secure left and right round pockets (12, 14) relative to elongate pocket (16). Likewise, loops (58) of elongate pocket (16) may selectively catch the hooks (not shown) of elongate fastening strap (not shown) if applicable to thereby selectively couple elongate pocket (16) to elongate fastening strap (not shown). In one example, loops (44, 58) of left and right round cooling capsules (22, 24) may catch corresponding hooks (not shown) provided within left and right interior cavities (32) of left and right round pockets (12, 14), respectively, to thereby selectively couple left and right round cooling capsules (22, 24) to left and right round pockets (12, 14), respectively. Such selective couplings between the components of modular cooling pack (10) may permit modular cooling pack (10) to be configurable in a variety of different configurations, each of which may be suitable for promoting particular types of pain relief, including facial healing.

### II. Method of Using a Modular Cooling Pack for Facial Healing

Referring now to FIGS. 18A-20B, a user, such as a clinician or patient, may use the modular cooling pack (10) by applying modular cooling pack (10) to a head (100) of a patient (102), such as to a face (104) of the patient (102) in any of a variety of possible configurations depending on the desired type of relief, including pain relief and facial healing, to be promoted.

As shown in FIGS. 1 and 5A, modular cooling pack (10) is configured in a first exemplary assembled configuration wherein left and right round cooling capsules (22, 24) are selectively retained within left and right round pockets (12, 14) and left and right round pockets (12, 14) are selectively coupled to elongate pocket (16) at or near respective left and right terminal end portions (66, 68) thereof. In addition, securement strap (18) compresses left and right round pockets (12, 14) with left and right round cooling capsules (22, 24) respectively inward toward left and right ears of the head (100). Also in the present example, securement strap (18) further compresses left and right terminal end portions (66, 68) inward respectively against left and right round pockets (12, 14) and toward left and right ears of the head (100). Thus, modular cooling pack (10) is worn by the patient (102) in the first exemplary assembled configuration such that left and right round pockets (12, 14) are secured to respective sides of the face (104) proximate an upper jaw (105) of the head (100) while elongate pocket (16) extends across the face (104) between a nose (108) and a mouth (110) of the head (100) without blocking, such as covering, openings in the mouth (110) and nose (108). In turn, modular cooling pack (10) cools the face (104) in the first exemplary assembled configuration without interfering with the patient's ability to breathe through the mouth (108) or nose (108). In this manner, left and right round cooling capsules (22, 24) applies localized cold temperatures to the upper jaw (105), such as for promoting facial healing during upper jaw surgery recovery. In one example, elongate cooling capsule (25) may be omitted from elongate pocket (16) such that cold temperatures are not applied to the portions of the patient's face contacted thereby when modular cooling pack (10) is in the first exemplary assembled configuration. In other words, elongate pocket (16) may simply function in a connecting capacity as desired or, alternatively may be completely omitted and only securement strap (18) is positioned against left and right round pockets (12, 14).

As shown in FIGS. 1 and 5B, modular cooling pack (10) is configured in a second exemplary assembled configuration similar to first exemplary assembled configuration except that rather than elongate pocket (16) being positioned between the nose (108) and the mouth (110), elongate pocket (16) with elongate cooling capsule (25) is secured over a dorsum of the nose (108). In this respect, elongate pocket (16) with elongate cooling capsule (25) does not block, such as cover, openings in the mouth (110) and nose (108) such that modular cooling pack (10) cools the face (104) in the second exemplary assembled configuration without interfering with the patient's ability to breathe through the mouth (108) or nose (108). Elongate cooling capsule (25) thus applies localized cold temperatures to the nose (108), such as for promoting facial healing during rhinoplasty recovery. As shown, left and right round pockets (12, 14) and left and right round cooling capsules (22, 24) may be omitted from modular cooling pack (10) when in the second assembled configuration.

As shown in FIGS. 1 and 5C, modular cooling pack (10) is configured in a third exemplary assembled configuration similar to first exemplary assembled configuration except that elongate pocket (16) and elongate cooling capsule (25) are omitted in such assembly. In other words, third exemplary assembled configuration only includes left and right round pockets (12, 14) with left and right cooling capsules (22, 24) and securement strap (18). Thus, modular cooling pack (10) may be worn by the patient (102) in the third exemplary assembled configuration such that left and right round pockets (12, 14) are secured to respective sides of the face (104) proximate to a posterior dentition of the patient (102). In this manner, left and right round cooling capsules (22, 24) may apply localized cold temperatures to the posterior dentition, such as for promoting facial healing during wisdom tooth removal recovery.

With respect to FIGS. 1 and 5D, a fourth exemplary assembled configuration only includes left and right round pockets (12, 14) with left and right cooling capsules (22, 24) and securement strap (18) such that elongate pocket (16) and elongate cooling capsule (25) are omitted in such assembly. Notably, securement strap (18) in the fourth exemplary assembled configuration extends around a circumference of the head (100) from approximately a crown at the rear of the head (100), around temples of the head (100), and about the forehead of the head (100). Left and right round pockets (12, 14) with left and right cooling capsules (22, 24) are thus positioned in the present example against the forehead of the head (100) and secured thereagainst by securement strap (18) for cooling pain relief, such as in the case of headaches, including migraines. In another example, one or more of left and right round pockets (12, 14) with left and right cooling capsules (22, 24) may be alternatively positioned anywhere between the head (100) and securement strap (18) to apply cooling temperatures to the crown, temples, and forehead as desired.

It will be appreciated that modular cooling pack (10) may be reusable and reconfigurable between its various configurations. Moreover, modular cooling pack (10) may be configurable in other configurations in addition to those shown and described herein. For example, modular cooling pack (10) may include additional round or elongate pockets, and/or pockets having other shapes and/or sizes which may be selectively coupled to each other and/or to the other components of modular cooling pack (10) to provide a desired, customized configuration for promoting pain relief, including facial healing.

### III. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A kit of a modular cooling pack for patient pain relief, comprising:
a first pocket;
a first cooling capsule configured to be received within the first pocket, wherein the first cooling capsule is configured to be cooled and applied to a head of a patient;
a second pocket;
a second cooling capsule configured to be received within the second pocket, wherein the second cooling capsule is configured to be cooled and applied to the head of the patient;
a third pocket;
a third cooling capsule configured to be received within the third pocket, wherein the third cooling capsule is configured to be cooled and applied to the head of the patient; and
a securement strap configured to receive the first, second, and third pockets thereagainst, wherein the securement strap at least partially defines a closed loop configured to be received about the head of the patient to thereby removably secure the first pocket, the second pocket, and the third pocket against the head of the patient in at least one of a plurality of assembled configurations.

2. The kit of claim 1, wherein the third pocket is elongated extending from a first terminal end portion to a second terminal end portion, wherein the first and second terminal end portions are configured to be received against the securement strap, and wherein the third cooling capsule is elongated so as to be complementarily shaped to the third pocket.

3. The kit of claim 1, wherein the first cooling capsule is complementarily shaped to the first pocket, and wherein the second cooling capsule is complementarily shaped to the second pocket.

4. The kit of claim 3, wherein each of the first cooling capsule, the first pocket, the second cooling capsule, and the second pocket is round.

5. The kit of claim 4, wherein the third pocket is elongated extending from a first terminal end portion to a second terminal end portion, wherein the first and second terminal end portions are configured to be received against the securement strap, and wherein the third cooling capsule is elongated so as to be complementarily shaped to the third pocket.

6. The kit of claim 1, wherein the securement strap has a band body defining a closed loop, wherein the band body is flexible, elastically resilient, and configured to stretch about the head of the patient and resiliently compress each of the first, second, and third pockets respectively with the first, second, and third cooling capsules against the head of the patient.

7. The kit of claim 6, wherein the band body has an inner surface defining a circumferential length about the closed loop and a width of the closed loop, wherein the circumferential length is larger than the width.

8. The kit of claim 1, wherein the first pocket includes a first interior cavity and a first closure flap such that the first closure flap is configured to cover at least a portion of the first interior cavity for retaining the first cooling capsule within the first interior cavity.

9. The kit of claim 8, wherein the second pocket includes a second interior cavity and a second closure flap such that the second closure flap is configured to cover at least a portion of the second interior cavity for retaining the second cooling capsule within the second interior cavity.

10. The kit of claim 9, wherein the third pocket includes a third interior cavity and a third closure flap such that the third closure flap is configured to cover at least a portion of the third interior cavity for retaining the third cooling capsule within the third interior cavity.

11. The kit of claim 1, wherein each of the first pocket, the second pocket, the third pocket, and the securement strap is separate from and not connected to each other for selective application to the head of the patient.

12. A method of forming a kit of a modular cooling pack for patient pain relief, the kit including a first pocket; a first cooling capsule configured to be received within the first pocket, wherein the first cooling capsule is configured to be cooled and applied to a head of a patient; a second pocket; a second cooling capsule configured to be received within the second pocket, wherein the second cooling capsule is configured to be cooled and applied to the head of the patient; a third pocket; a third cooling capsule configured to be received within the third pocket, wherein the third cooling capsule is configured to be cooled and applied to the head of the patient; and a securement strap configured to receive the first, second, and third pockets thereagainst, wherein the securement strap at least partially defines a closed loop configured to be received about the head of the patient to thereby removably secure the first pocket, the second pocket, and the third pocket against the head of the patient in at least one of a plurality of assembled configurations, the method comprising:
collecting each of the first pocket, the second pocket, the third pocket, the first cooling capsule, the second cooling capsule, the third cooling capsule, and the securement strap together into a packaging and forming the kit of the module cooling pack.

13. A method of cooling a head of a patient with a modular cooling pack, wherein the modular cooling pack includes a first pocket; a first cooling capsule configured to be received within the first pocket, wherein the first cooling capsule is configured to be cooled and applied to the head of the patient; a second pocket; a second cooling capsule configured to be received within the second pocket, wherein the second cooling capsule is configured to be cooled and applied to the head of the patient; and a securement strap configured to receive the first and second pockets thereagainst, wherein the securement strap at least partially defines a closed loop configured to be received about the head of the patient to thereby removably secure the first pocket and the second pocket against the head of the patient, the method comprising:
surrounding the head of the patient with at least the securement strap thereby compressing the first pocket, the first cooling capsule, the second pocket, and the second cooling capsule against the head of the patient such that neither the first pocket nor the second pocket block breathing through a nose or a mouth of the patient.

14. The method of claim 13, wherein the module cooling pack further includes a third pocket and a third cooling capsule, wherein the third cooling capsule is configured to be received within the third pocket, wherein the third cooling capsule is configured to be cooled and applied to the head of the patient.

15. The method of claim 14, further comprising releasably securing the third pocket to the first and second pocket such that the third pocket extends across the face of the patient such that the third pocket does not block breathing through the nose nor the mouth of the patient.

16. The method of claim 15, wherein the third pocket is positioned between the nose and the mouth of the patient.

17. The method of claim 15, wherein the third pocket is positioned on a dorsum of the nose of the patient.

18. The method of claim 15, wherein the third pocket has a first terminal end and a second terminal end, and wherein each of the first and second terminal ends are compressed toward the head of the patient via the securement strap.

19. The method of claim 15, wherein the securement band extends from under a lower jaw of the head to an opposing top of the head.

20. The method of claim 13, wherein the securement band extends from a crown of the head to an opposing forehead of the head.
